(19) United States
(12) Patent Application Publication
(10) Pub. No.: US 2022/0101958 A1
(43) Pub. Date: Mar. 31, 2022

(54) DETERMINATION OF IMAGE STUDY ELIGIBILITY FOR AUTONOMOUS INTERPRETATION

(71) Applicant: KONINKLIJKE PHILIPS N.V., EINDHOvEN (NL)

(72) Inventor: MERLIJN SEVENSTRA, HAARLEM (NL)

(21) Appl. No.: 17/439,842

(22) PCT Filed: Mar. 18, 2020
(86) PCT No.: PCT/EP2020/057465

§ 371 Date: Sep. 16, 2021

Related U.S. Application Data
(60) Provisional application No. 62/820,880, filed on Mar. 20, 2019.

(51) Int. Cl. G16H 10/20; G06T 7/00; G16H 30/20

(52) U.S. Cl. CPC G16H 10/20; G06T 7/0014; G06T 2207/20084; G06T 2207/30004; G16H 30/20

(57) ABSTRACT

A system and method for determining whether an image study is eligible for autonomous interpretation. The method includes detecting a likelihood assessment of whether a particular pathology is present in a current image study using an AI model for the particular pathology. The method includes electing a relevant prior image study that has been assessed via the AI model. The method includes retrieving relevant information pertaining to one of the current image study and the relevant prior image study. The method includes determining, based on at least one of the likelihood assessment of the current image study, the relevant prior image study and the retrieved relevant information, whether the current image study is eligible for autonomous interpretation.

### BACKGROUND

It is anticipated that autonomous Artificial Intelligence (AI) will play an increasingly important role in healthcare. Radiological studies, in particular, have generally required a radiologist to interpret the image studies. In some cases, however, radiologists may add little to no value as highly trained and costly human resources. For example, interpretation of normal or stable chest x-rays may not require the same level of expertise as more complex studies. Thus, normal or stable chest x-rays may be good candidates for autonomous interpretation, thereby reducing workload for radiologists.

Current automated diagnostic systems which use, for example, machine learning, are impressive, but cannot correctly diagnose pathologies on each and every image study. For example, the automated diagnosis of one chest x-ray requires assessment of the image for over twenty pathologies. Even with highly accurate diagnostic models with a sensitivity of 0.999, for each of the over 20 pathologies, the cumulative error of missing at least one pathology is 0.98 (i.e., 2 in 100). This rate may not be acceptable in a general clinical setting, while being difficult to improve.

Thus, the possible use of AI to analyze image studies such as x-rays produces a need to identify the image studies that are acceptable candidates to be analyzed by AI.

### SUMMARY

The exemplary embodiments are directed to a method, comprising: detecting a likelihood assessment of whether a particular pathology is present in a current image study using an AI model for the particular pathology; selecting a relevant prior image study that has been assessed via the AI model; retrieving relevant information pertaining to one of the current image study and the relevant prior image study; and determining, based on at least one of the likelihood assessment of the current image study, the relevant prior image study and the retrieved relevant information, whether the current image study is eligible for autonomous interpretation.

The exemplary embodiments are directed to a system, comprising: a non-transitory computer readable storage medium storing an executable program; and a processor executing the executable program to cause the processor to: detect a likelihood assessment of whether a particular pathology is present in a current image study using an AI model for the particular pathology; select a relevant prior image study that has been assessed via the AI model; retrieve relevant information pertaining to one of the current image study and the relevant prior image study; and determine, based on at least one of the likelihood assessment of the current image study, the relevant prior image study and the retrieved relevant information, whether the current image study is eligible for autonomous interpretation.

The exemplary embodiments are directed to a non-transitory computer-readable storage medium including a set of instructions executable by a processor, the set of instructions, when executed by the processor, causing the processor to perform operations, comprising: detecting a likelihood assessment of whether a particular pathology is present in a current image study using an AI model for the particular pathology; selecting a relevant prior image study that has been assessed via the AI model; retrieving relevant information pertaining to one of the current image study and the relevant prior image study; and determining, based on at least one of the likelihood assessment of the current image study, the relevant prior image study and the retrieved relevant information, whether the current image study is eligible for autonomous interpretation.

### BRIEF DESCRIPTION OF THE DRAWINGS

FIG. 1 shows a schematic drawing of a system according to an exemplary embodiment.

FIG. 2 shows a further schematic diagram of the system according to FIG. 1.

FIG. 3 shows a flow diagram of a method for comparing an AI assessment and a radiological report for an image study according to an exemplary embodiment.

FIG. 4 shows a flow diagram of a method for determining whether an image study is eligible to be autonomously interpreted.

### DETAILED DESCRIPTION

The exemplary embodiments may be further understood with reference to the following description and the appended drawings, wherein like elements are referred to with the same reference numerals. The exemplary embodiments relate to systems and methods for determining whether a particular image study qualifies for autonomous interpretation. The exemplary embodiments improve the operation of automated diagnostic systems by identifying those studies (e.g., normal or stable chest x-rays) that can be accurately interpreted by the automated system, leaving any remaining studies to be read and interpreted by a radiologist. Thus, pathologies for all image studies are interpreted with greater accuracy and precision while also reducing workload for the radiologist. It will be understood by those of skill in the art that although the exemplary embodiments are shown and described with respect to chest x-rays, the systems and methods of the present disclosure may be similarly applied to any of a variety of radiological studies.

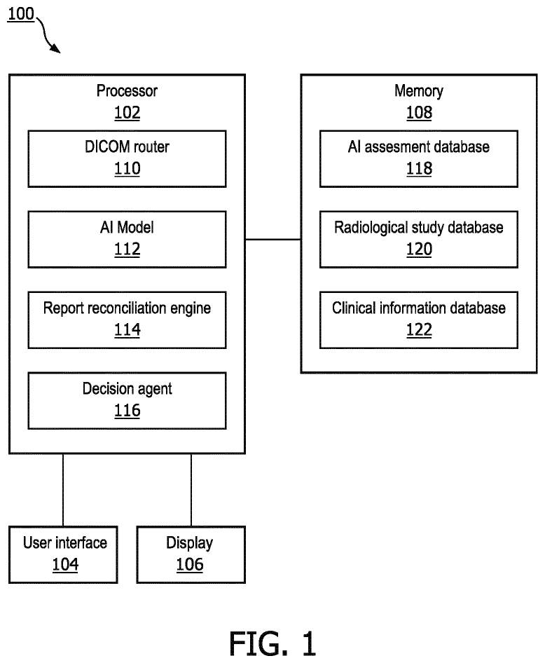

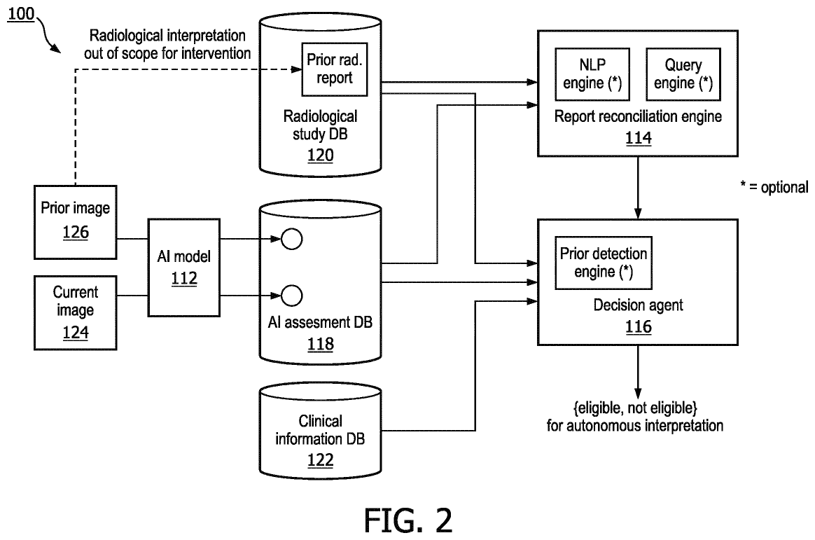

As shown in FIGS. 1 and 2, a system 100, according to an exemplary embodiment of the present disclosure, determines whether a current image study 124 qualifies and/or is eligible for a fully autonomous interpretation. The system 100, as shown in FIG. 1, comprises a processor 102, a user interface 104, a display 106, and a memory 108. The processor 102 may include or execute a DICOM (Digital Imaging and Communications in Medicine) router 110, an AI model 112, a report reconciliation engine 114 and a decision agent 116. The memory 108 may include an AI assessment database 118, a radiological study database 120 and a clinical information database 122.

As shown in FIG. 2, the DICOM router 110 directs a recently acquired current image study 124 to the AI Model 112, which automatically assesses the current image study 124 for a given pathology. The AI model assessment is stored in the AI assessment database 118. Prior image studies 126 from the radiological study database 120, which have already been interpreted by a radiologist and thereby include radiology reports, are also assessed via the AI Model 112 and stored in the AI assessment database 118. The report reconciliation engine 114 determines if reports of each of the prior image studies are in agreement with the corresponding AI assessments. Based on output from the AI model 112 for the current image study 124, AI model 112 and report reconciliation engine 114 output for an identified relevant prior image study, and/or relevant patient information from the clinical information database 122, the decision agent 116 determines whether the current image study 124 may be autonomously interpreted.

The AI model 112 may be created with machine learning or image processing techniques to detect one specific pathology. In particular, the AI model 112 provides an assessment of the likelihood of a presence of the modeled pathology. The AI model 112 may also mark individual pixels/voxels on the current image study 124 as a “heat map” indicative of the detected pathological process. For example, individual pixels/voxels may include color-coded markings to indicate a particular pathology. The current image study 124 may be subsequently displayed with these markings to a user (e.g., radiologist) on the display 106. The user may then select any one of these markings via the user interface 104 for further information regarding the identified pathology associated with the marking. Although the exemplary embodiments show and describe a single AI model 112, it will be understood by those of skill in the art that the system 100 may include a variety of AI models, each of which are optimized for detecting one specific pathology. For chest X-rays, for example, more than 20 different AI models 112 may be included to detect the more than 20 different potential pathologies identifiable in a chest x-ray. These pathologies may include, for example, tuberculosis, edema, lung nodules, emphysema, fractured ribs, etc. It should also be understood that in some exemplary embodiments, an AI model may be optimized for more than a single pathology.

The report reconciliation engine 114 compares the AI assessment of the AI model 112 for prior image studies with the radiology reports thereof by normalizing the assessment in the radiology report to the same scale. For example, for free-text radiology reports, a natural language processing (NLP) module may be optimized to detect pathologies and the status. The NLP module may utilize string-matching techniques and keywords indicative of certainty (e.g., there is no evidence of, cannot be excluded, etc.). For semi-structured radiology reports (e.g., eXtensible Markup Language (XML) format), the report reconciliation engine 114 may utilize a querying engine to query structured content using a formal query language (e.g., xPath). The normalized assessment of the radiology report may then be compared to the AI assessment to determine whether the AI assessment and the radiology report are in agreement. These prior image studies and their corresponding AI and report reconciliation assessments may be similarly stored to the AI assessment database 118.

The decision agent 116 retrieves the AI assessment for the current image study 124 and the AI assessment of one or more of the relevant prior image studies 126 based on, for example, study date (e.g., within 30 days), indication, anatomy, and/or modality. The decision agent 116 may also retrieve radiological study data (e.g., indicator if the study is ER/inpatient/outpatient, ordering physician) from the radiological study database 120 and/or clinical information (e.g., age, any recent new diagnoses, etc.). All of the retrieved information may be analyzed, as will be described in further detail below, to determine whether the current image study may be autonomously interpreted.

Those skilled in the art will understand that the DICOM router 110, the AI model 112, the report reconciliation engine 114 and the decision agent 116 may be implemented by the processor 102 as, for example, lines of code that are executed by the processor 102, as firmware executed by the processor 102, as a function of the processor 102 being an application specific integrated circuit (ASIC), etc. It will also be understood by those of skill in the art that although the system 100 is shown and described as comprising a computing system comprising a single processor 102, user interface 104, display 106 and memory 108, the system 100 may be comprised of a network of computing systems, each of which includes one or more of the components described above. In one example, the DICOM router 110, the AI model 112, the report reconciliation engine 114 and the decision agent 116 may be executed via a central processor of a network, which is accessible via a number of different user stations. Alternatively, one or more of the DICOM router 110, AI model 112, report reconciliation engine 114 and the decision agent 116 may be executed via one or more processors. Similarly, the radiology study database 118, the AI assessment database 120 and the clinical information database 122 may be stored to a central memory 108 or, alternatively, to one or more remote and/or network memories 108.

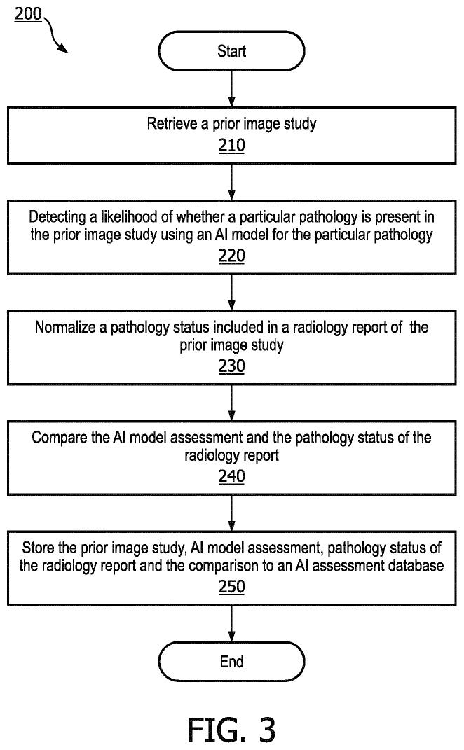

FIG. 3 shows an exemplary method 200 for providing an AI assessment of prior image studies 126 and comparing the AI assessment of each of the prior image studies with a corresponding radiology report thereof. In 210, a prior image study 126 that has been previously interpreted by a radiologist is retrieved from the radiological study database 120 and transmitted to the AI model 112. In 220, the AI model 112 assesses the prior image study 126 to detect a particular pathology. If the prior image study 126 has more than one series, the AI model 112 may be applied to either a subset of the series or all of the series. The AI model 112 returns a likelihood assessment—indicating a likelihood of existence of the particular pathology based on the prior image study 126—in the range of [0,1], 0 representing the particular modeled pathology definitely not being present and 1 representing the modeled pathology definitely being present. The AI assessment, which includes the likelihood assessment described above, may be stored to the AI assessment database 118. The AI model may further mark individual pixels/voxels on the prior image study 126 indicative of the detected pathology.

In 230, the report reconciliation engine 114 normalizes the pathology status included in the radiology report of the prior image study 126 to the same scale as the AI assessment. In one example, for free text radiology reports, an NLP module can use string matching techniques to detect mentions of the modeled pathology and certainty keywords. The search mechanism may be configured such that it accounts for lexical variants and abbreviations. Techniques may be used to derive whether a pathology is within the scope of a detected keyword to assess the status of the pathology. Using, for example, a mapping table, the keywords may be mapped onto a five-point scale, e.g., where 1 indicates the strongest radiological evidence for presence and 5 indicates no radiological evidence for presence. This scale may be simplified by mapping, for example, the five-point scale to a two-point scale using predetermined mapping. A dedicated value may be used to indicate that the pathology was not mentioned in the report and/or that its reported status was unclear. Thus, the NLP module is able to derive, for a series of pathologies, the reported status in a radiology report on a normalized scale.

In another example, for a semi-structured radiology report, structured content may be converted into human-understandable free text for inclusion into the radiology report. The structured content may be queried using a formal query language. If the structured content has elements for encoding pathologies and their status, this can be retrieved from the structured content directly, producing an output that may be made consistent with the normalized scale that is described above with respect to the free text radiology report.

In 240, the AI assessment derived in 220 (e.g., a core in the range [0,1]), and the semantically normalized assessment from the radiology report (e.g., a score on a five-point scale) are compared. These two scales may be compared using, for example, a mapping table in which scale item 1 maps onto a certainty range [0,0.2], etc. The radiology report and the AI may thus be considered to be in agreement on a pathology if the AI assessment falls within the range of the report certainty marker.

210-240 may be repeated for each of the AI models 112 modeling a different pathology to identify and compare the AI assessment and radiology report for each of the modeled pathologies. In 250, the report reconciliation engine 114 determines whether the radiology report and the AI models are in agreement on the given prior image study 126—i.e., if they are in agreement on all of the pathologies detected by the different AI models. Based on this assessment, the report reconciliation engine 114 may return values:
Agreement on all AI-detected pathologies (a)
Disagreement on at least one AI-detected pathology (D1)
Disagreement on X AI-detected pathologies (D2)

Where the NLP module (or query language) detects more pathologies that the AI models, the report reconciliation engine 114 may return the following:
Agreement on all AI-detected pathologies, and all non-AI-detected pathologies are reported normal (AN)
Agreement on all AI-detected pathologies, and there is at least one non-AI detected pathology report abnormal (AA)

Thus, the report reconciliation may return codes A, D1, D2, AN, and/or AA. It will be understood by those of skill in the art, however, that these codes are exemplary only and that the report reconciliation engine 114 may output other codes and/or additional codes to represent the comparison results.

In 260, the prior image study 126 and the AI assessment and radiology assessment may be stored in the AI assessment database 118. Although storage in the assessment database 118 is described and shown as taking place in the step 260, it will be understood by those of skill in the art that these assessments may be stored in the AI assessment database 118 at any time during the method 200. It will also be understood by those of skill in the art that the method 200 is repeated for a multitude of prior image studies stored in the radiological study database 120.

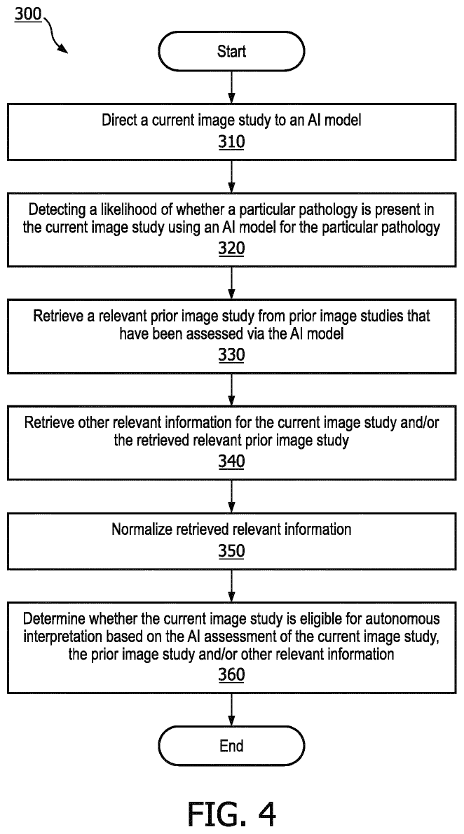

FIG. 4 shows a method 300 utilizing the system 100 for determining whether a current image 124 is qualified to be autonomously interpreted, as will be described in further detail below. In 310, the DICOM router 110 (or “sniffer”) catches the current image study 124 (e.g., a recently acquired DICOM study) while it is being sent from the modality (e.g., x-ray, MRI, ultrasound, etc.) to the Picture Archiving and Communications Systems (PACS) and directs the current image study 124 to the AI model 112.

In 320, the AI model 112 assesses the current image study 124 to detect a particular pathology. The AI model 112 returns a likelihood assessment—indicating a likelihood of existence of the particular pathology based on the current image study 124—in the range of [0,1], 0 representing the particular modeled pathology definitely not being present and 1 representing the modeled pathology definitely being present. If the current image study 124 has more than one series, the AI model 112 may be applied to either a subset of the series or all of the series. The AI assessment, which includes the likelihood assessment described above, may be stored to the AI assessment database 118. The AI model 112 may further mark individual pixels/voxels on the current image study 126 indicative of the detected pathology. The AI assessment of the current image study 124 may be stored in the AI assessment database 118. As discussed above, although the exemplary embodiment shows and describes one AI model 112 which detects a particular pathology, the system 100 may include a plurality of AI models, each of which detects a different pathology. Thus, it will be understood by those of skill in the art, that 320 may be repeated for each modeled pathology.

In 330, the decision agent 116 retrieves an AI assessment for one or more relevant prior image studies that have been stored on the AI assessment database, as described above with respect to the method 200. Relevancy is determined by information retrieved from the radiological study database 120 and may be based on comparison study date (e.g., within 30 days), indication, anatomy and/or modality. In one embodiment, the decision agent may identify a most relevant prior image study using rule-based logic taking into account modality and field of view similarity. Using string matching and concept matching techniques, for example, lexically different but semantically matching strings can be resolved.

In 340, the decision agent 116 retrieves any relevant information that may be used to determine whether the current image study 124 qualifies for autonomous interpretation. Relevant information may include information such as, for example, radiological study data from the radiological study database 120 for the current image study 124 and the relevant prior image study retrieved in 330. Study data may include information such as, for example, an indicator if the study is ER, outpatient or inpatient, and the ordering physician. Relevant information may also include clinical information for the patient of the current image study 124 from the clinical information database 122. Clinical information may include information such as, for example, patient age (e.g., whether the patient is pediatric/adult), any recent new diagnoses, etc. In 350, retrieved relevant information may be normalized into binary variables (e.g., pediatric=0, adult=1) using standard mapping tables of information.

In 360, the decision agent determines whether the current image study 124 qualifies for autonomous interpretation based on the AI assessment of the current image study 124, the relevant prior image study and the relevant information retrieved in 340. The decision agent may apply, for example, rule-based logic and/or subsymbolic reasoning (e.g., based on a neural network or logistic regression model) to come to an assessment if the DICOM study can be interpreted autonomously. Rules used by the decision agent 116 may include, for example:
If the patient of the current image study 124 is pediatric, then the current image study 124 is NOT ELIGIBLE for autonomous interpretation.
If the current image study 124 was ordered by the ER, then the current image study 124 is NOT ELIGIBLE for autonomous interpretation.
If the AI assessment of the current image study 124 does not match the AI assessment of the relevant prior image study, then the current image study 124 is NOT ELIGIBLE for autonomous interpretation.
IF the AI assessment of the relevant prior image study did not match the radiological report of the relevant prior image study, then the current image study 124 is NOT ELIGIBLE for autonomous interpretation.
If the patient of the current image study 124 has an active diagnosis on his/her problem list that was added since the most recent related prior study, then the current image study 124 is NOT ELIGIBLE for autonomous interpretation.
If none of the above, then the current image study 124 is ELIGIBLE for autonomous interpretation.

It will be understood by those of skill in the art that the above rules are exemplary only and that the decision agent 116 may utilize one or more of the rules above and/or other rules to determine, for example, the stability of the current image study 124 and whether it is eligible for autonomous interpretation. In another embodiment, the last rule noted above may be replaced by a rule that calls a subsymbolic reasoner. For example, the decision agent 116 may employ the rule:
If none of the above, then call the neural network based on the various outputs and return an output based on an eligibility likelihood.

If the eligibility likelihood includes a binary output, for example, the decision agent 116 may utilize a predefined threshold to interpret the likelihood output (e.g., 0.0.5 indicates eligibility).

Those skilled in the art will understand that the above-described exemplary embodiments may be implemented in any number of manners, including, as a separate software module, as a combination of hardware and software, etc. For example, the DICOM router 110, the AI model 112, the report reconciliation engine 114 and the decision agent 116 may be programs containing lines of code that, when compiled, may be executed on the processor 102.

It will be apparent to those skilled in the art that various modifications may be made to the disclosed exemplary embodiments and methods and alternatives without departing from the spirit or scope of the disclosure. Thus, it is intended that the present disclosure cover the modifications and variations provided that they come within the scope of the appended claims and their equivalents.

1. A computer-implemented method, comprising:
detecting a likelihood assessment of whether a particular pathology is present in a current image study using an AI model for the particular pathology;
selecting a relevant prior image study that has been assessed via the AI model;
retrieving relevant information pertaining to one of the current image study and the relevant prior image study; and
determining, based on at least one of the likelihood assessment of the current image study, the relevant prior image study and the retrieved relevant information, whether the current image study is eligible for autonomous interpretation;
wherein determining whether the current image is not eligible for autonomous interpretation includes deploying a set of rules including a rule which states that, if an AI assessment of the relevant prior image study did not match a radiological report of the relevant prior image study, then the current study is not eligible for autonomous interpretation.

2. The method of claim 1, further comprising:
detecting, for each of a plurality of prior image studies that have been previously interpreted, a likelihood assessment of whether a particular pathology is present in the plurality of prior image studies;
normalizing a pathology status included in a radiology report of each of the plurality of prior studies;
comparing the likelihood assessment and the normalized pathology status of the radiology report for each of the plurality of prior image studies to determine whether the likelihood assessment and the radiology report are in agreement; and
storing the plurality of prior image studies and each of the corresponding likelihood assessments and results of comparison between the likelihood assessment and the radiology report to AI assessment database.

3. The method of claim 2, wherein the relevant prior image study is selected from one of the plurality of prior image studies.

4. The method of claim 1, wherein the relevant prior image study is selected based on a commonality between the current image study and the relevant prior image study of at least one of a study date, indication, anatomy and modality.

5. The method of claim 1, wherein the retrieved relevant information includes at least one of radiological study data and clinical information for a patient of the current image data.

6. The method of claim 1, further comprising normalizing the retrieved relevant information.

7. The method of claim 1, the set of rules further including at least one of:
(a) if the patient is pediatric, then the current image study not eligible;
(b) if the likelihood assessment of the current image study does not match the likelihood assessment of the relevant prior image study, then the current image study is not eligible;
(c) if a patient of the current image study has an active diagnosis, then the current image study is not eligible; and
(d) if the current image study was ordered by a user in an ER department, the current image study is not eligible.

8. The method of claim 7, wherein, if it is determined the set of rules for determining non-eligibility are not met, it is determined that the current image study is eligible for autonomous interpretation.

9. The method of claim 7, wherein, if it is determined that the set of rules for non-eligibility are not met, the method further comprises calling a neural network and determining a likelihood of eligibility score.

10. The method of claim 9, wherein a predetermined threshold s used to determine eligibility based on the likelihood of eligibility score.

11. A system, comprising:
a non-transitory computer readable storage medium storing an executable program; and
a processor executing the executable program to cause the processor to perform the method of claim 1.

12. (canceled)

13. The system of claim 11, further comprising an AI assessment database storing one of the likelihood assessment of the current image study and the plurality of prior image studies and each of the corresponding likelihood assessments and results of comparison between the likelihood assessment and the radiology report to the AI assessment database.

14.-19. (canceled)

20. A non-transitory computer-readable storage medium including a set of instructions executable by a processor, the set of instructions, when executed by the processor, causing the processor to perform the method of claim 1.
